(19) United States
(12) Patent Application Publication
(10) Pub. No.: US 2024/0225619 A1
(43) Pub. Date: Jul. 11, 2024

(54) TISSUE SAMPLE DEVICE AND METHODS

(71) Applicant: Boston Scientific Scimed, Inc., Maple Grove, MN (US)

(72) Inventors: Peter L. Dayton, Brookline, MA (US); John B. Golden, Norton, MA (US); James Weldon, Newton, MA (US)

(73) Assignee: Boston Scientific Scimed, Inc., Maple Grove, MN (US)

(21) Appl. No.: 18/409,271

(22) Filed: Jan. 10, 2024

Related U.S. Application Data
(60) Provisional application No. 63/438,381, filed on Jan. 11, 2023.

(51) Int. Cl. A61B 10/02

(52) U.S. Cl. CPC A61B 10/02; A61B 2010/0216; A61B 2217/005

(57) ABSTRACT

A tissue collection system for collecting a sample from the body. The system may comprise a tubular member having a proximal end region and a distal end region and defining a lumen extending from the proximal end region to the distal end region, an elongate shaft slidably disposed within the lumen of the tubular member, the elongate shaft extending from a proximal end region to a distal end region, a tissue collection device disposed adjacent to the distal end region of the elongate shaft, and a filter positioned proximal to a distal end region of the tissue collection device when the tissue collection device is in a use configuration.

### CROSS-REFERENCE TO RELATED APPLICATIONS

This application claims the benefit of priority under 35 U.S.C. § 119 of U.S. Provisional Application No. 63/438,381, filed Jan. 11, 2023, the entire disclosure of which is hereby incorporated by reference.

### TECHNOLOGY FIELD

The present application relates generally to medical devices for tissue sample collection, and more particularly, to medical devices for tissue sample collection resulting in greater cellular or other tissue yield.

### BACKGROUND

Certain medical tests require sampling of cells from target areas of a subject's body. For instance, a screening test for detecting potentially pre-cancerous and cancerous tissues in a subject's body may include taking samples of tissue or cells from a target area of the subject's body. A tissue collection device may be used to collect cells or other tissues from the target area. Tissue collection from some parts of the anatomy may be difficult. There is an ongoing need to provide alternative medical devices as well as alternative methods for manufacturing and using medical devices.

### SUMMARY

This disclosure provides design, material, manufacturing methods, and use alternatives for medical devices.

In a first example, a tissue collection system may comprise a tubular member having a proximal end region and a distal end region and defining a lumen extending from the proximal end region to the distal end region, an elongate shaft slidably disposed within the lumen of the tubular member, the elongate shaft extending from a proximal end region to a distal end region a tissue collection device disposed adjacent to the distal end region of the elongate shaft, and a filter positioned proximal to a distal end region of the tissue collection device when the tissue collection device is in a use configuration.

Alternatively or additionally to any of the examples above, in another example, the filter may include a plurality of pores.

Alternatively or additionally to any of the examples above, in another example, a pore size of the plurality of pores may decrease from a distal end to a proximal end of the filter.

Alternatively or additionally to any of the examples above, in another example, the filter may be disposed within the lumen of the tubular member.

Alternatively or additionally to any of the examples above, in another example, the filter may define a lumen extending from a proximal end to a distal end thereof.

Alternatively or additionally to any of the examples above, in another example, the filter may be coupled to an inner surface of the tubular member.

Alternatively or additionally to any of the examples above, in another example, the tissue collection device may include a tissue collection member defining a cavity.

Alternatively or additionally to any of the examples above, in another example, the filter may be disposed within the cavity of the tissue collection member.

Alternatively or additionally to any of the examples above, in another example, the cavity of the tissue collection member may be in fluid communication with a lumen of the elongate shaft.

Alternatively or additionally to any of the examples above, in another example, the tissue collection system may further comprise one or more apertures formed through a sidewall of the tissue collection member.

Alternatively or additionally to any of the examples above, in another example, the filter may comprise a filter assembly affixed to a distal end region of the tubular member.

Alternatively or additionally to any of the examples above, in another example, the filter assembly may include an expandable frame and a filter element coupled to the expandable frame.

Alternatively or additionally to any of the examples above, in another example, the tissue collection system may further comprise an outer sheath, the tubular member slidably disposed within a lumen of the outer sheath.

Alternatively or additionally to any of the examples above, in another example, the tissue collection system may further comprise a vacuum source.

Alternatively or additionally to any of the examples above, in another example, the vacuum source may be configured to draw fluid and tissue through the filter.

In another example, a tissue collection system may comprise a tubular member having a proximal end region and a distal end region and defining a lumen extending from the proximal end region to the distal end region, an elongate shaft slidably disposed within the lumen of the tubular member, the elongate shaft extending from a proximal end region to a distal end region, a plurality of bristles disposed adjacent to the distal end region of the elongate shaft, and a filter positioned within the lumen of the tubular member, the filter configured to collect cells dislodged from a target collection site.

Alternatively or additionally to any of the examples above, in another example, the tissue collection system may further comprise a vacuum source fluidly coupled to the proximal end region of the tubular member.

Alternatively or additionally to any of the examples above, in another example, the vacuum source may comprise a syringe.

In another example, a tissue collection system may comprise an outer tubular member having a proximal end region and a distal end region and defining a lumen extending from the proximal end region to the distal end region and a tissue collection device. The tissue collection device may comprise an elongate tubular shaft slidably disposed within the lumen of the outer tubular member, the elongate tubular shaft extending from a proximal end region to a distal end region, a tissue collection member disposed on a distal end region of the elongate tubular shaft, the tissue collection member defining a cavity and having a plurality of apertures extending from an outer surface of the tissue collection member to the cavity, and a plurality of bristles extending radially from an outer surface of the tissue collection member. The tissue collection system may further comprise a filter positioned within the cavity of the tissue collection member, the filter configured to collect cells dislodged from a target collection site.

Alternatively or additionally to any of the examples above, in another example, the tissue collection system may further comprise a vacuum source fluidly coupled to the proximal end region of the elongate tubular shaft.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 illustrates a partial cross-sectional side view of an illustrative tissue collection device system for delivering a tissue collection device to a target region in a use configuration within a vessel;

FIG. 2 is a schematic partial cross-sectional view of a portion of the system of FIG. 1 coupled to a syringe;

FIG. 3 is a partial cross-sectional side view of another illustrative tissue collection device system for delivering a tissue collection device to a target region in a use configuration; and

FIG. 4 illustrates a partial cross-sectional side view of another illustrative tissue collection device system for delivering a tissue collection device to a target region in a use configuration within a vessel.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION OF EXEMPLARY EMBODIMENTS

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features, and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements but may include other elements not expressly listed or inherent to such process, method, article, or apparatus. Additionally, terms that indicate the geometric shape of a component/surface refer to exact and approximate shapes.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Endoscopic retrograde cholangiopancreatography (ERCP) is a procedure that utilizes both endoscopic and fluoroscopic techniques to diagnose and treat issues arising in the common bile duct (CBD) and pancreatic ducts (PD). One of the main issues being treated today is strictures in the CBD from such as, but not limited to, primary sclerosing cholangitis (PSC), cancer of the bile duct, damage and scarring due to a gallstone in the bile duct, etc. However, biliary structures may have a low cancer sensitivity rate which may lead to a false-negative diagnosis during an ERCP procedure. In some cases, low sensitivity may be linked to inadequate tissue sampling which is a primary limiting factor of detecting potential malignancy. When a clinician needs to take a sample of the stricture, one of the most common ways to do this is by using a cytology brush. However, the sensitivity of a cytology brush may be between about 30-60%. The sensitivity of cytology brushes may be mainly linked to insufficient quantities of samples. One of the reasons cytology brushes may not be able to collect enough samples is the use of soft bristles. Another reason may be losing samples during the procedure.

What may be desirable is a sample collection device that collects a sufficient amount of samples for better prediction of biliary cancer. While the present disclosure is described with respect to the common bile duct and pancreatic ducts, the devices and methods are not limited to such use. For example, the devices and methods described herein may be used in any portion of the anatomy, as desired. Further, the devices and methods described herein may be used either endoscopic or non-endoscopic anatomies. Some illustrative anatomies may include, but are not limited to, the mouth, the esophagus, the stomach, the duodenum, other portions of the gastrointestinal tract, the pathways leading to the lungs, other portions of the respiratory system, the urinary tract, the cervix, other reproductive anatomy, etc.

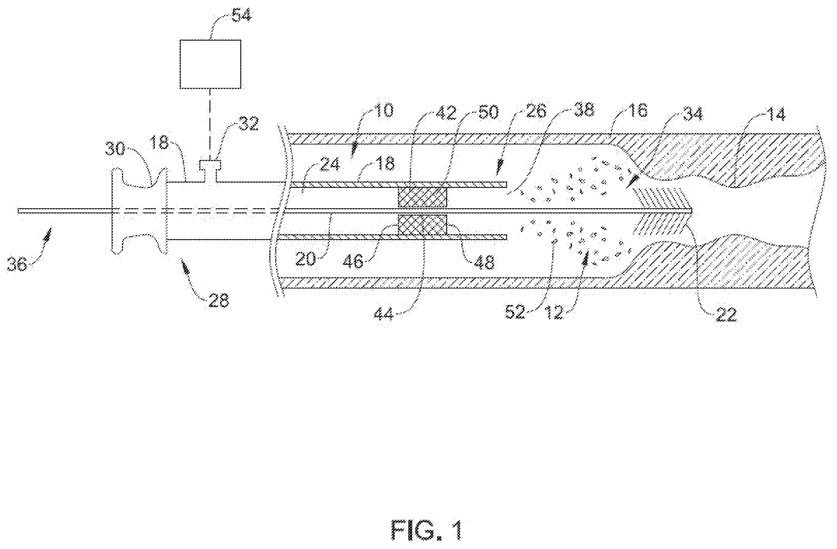

FIG. 1 is a partial cross-sectional side view of an illustrative tissue collection system 10 for delivering a tissue collection device 12 to a target region 14, such as, but not limited to, the common bile duct or pancreatic duct 16, with the tissue collection device 12 in an extended or use configuration. The tissue collection system 10 may include an outer or exterior elongate shaft or tubular member 18, such as, but not limited to, a catheter or introduction sheath. The system 10 may further include a tissue collection device 12 having an elongate shaft 20 and a plurality of bristles 22 disposed on a distal portion of the elongate shaft 20. The tissue collection device 12 may be slidably disposed within a lumen 24 of the outer tubular member 18.

The outer tubular member 18 may extend proximally from a distal end region 26 to a proximal end region 28 configured to remain outside of a patient's body. A first hub or handle 30 may be coupled to the proximal end region 28 of the outer tubular member 18. In some cases, a port 32, such as an injection port, may be provided in the outer tubular member 18. Other structures to facilitate connection to other medical devices (e.g., syringe, stopcocks, Y-adapter, etc.) and to provide access to lumen 24 may be provided. The elongate shaft 20 may extend proximally from a distal end region 34 to a proximal end region 36 configured to remain outside of a patient's body.

The outer tubular member 18 may include a lumen 24 extending from a distal opening 38 at the distal end region 26 to the proximal end region 28. The lumen 24 may also extend through the first handle 30. The lumen 24 of the outer tubular member 18 and the first handle 30 may be configured to slidably receive the elongate shaft 20. The lumen 24 of the outer tubular member 18 may also be configured to receive a guidewire (not explicitly shown). It is contemplated that the system 10 may be arranged such that the guidewire extends within a lumen along an entire length of the outer tubular member 18 in an “over-the-wire” manner or the guidewire may exit a side port in the outer tubular member 18 distal to the proximal end region 28 thereof in a “rapid-exchange” manner.

During insertion of the system 10 into the subject's body, or withdrawal of the system 10 from the subject's body, the tissue collection device 12 may be in a retracted position, with the elongate shaft 20 pulled proximally to position the bristles 22 within the lumen 24 of the outer tubular member 18. The elongate shaft 20 of the tissue collection device 12 may be pushed distally to move the tissue collection device to an extended position (shown in FIG. 1), with the bristles 22 extending out of the distal opening 38, so they are exposed from the outer sheath 18.

The elongate shaft 20 may include one or more wires twisted into an elongated form. The elongate shaft 20 may have sufficient flexibility to allow it to bend during insertion of the tissue collection device 12 into or withdrawal of the tissue collection device 12 from the subject's body. The elongate shaft 20 may have sufficient rigidity so that pushing or pulling of the elongate shaft 20 may cause extension or retraction, respectively, of the tissue collection device 12 from the outer tubular member 18. A proximal end region 36 of the elongate shaft 20 may be gripped by a user such that the user may manually push or pull the elongate shaft 20. Dimensions of the elongate shaft 20 may vary depending upon the subject's anatomy and/or the type of procedure being performed.

In some embodiments, the tissue collection device 12 may include a brush mechanism including a plurality of bristles 22. While the tissue collection device 12 is described as a brush mechanism, it is contemplated that other tissue collection mechanisms may be used, as desired. The bristles 22 may extend radially outward from an outer surface of the elongate shaft 20. The bristles 22 may be coupled, adhered, or otherwise affixed to surface of the elongate shaft 20. In some cases, the bristles 22 may be coupled directly to the elongate shaft 20. In some examples, the bristles 22 may be clamped between the twisted wires of the elongate shaft 20. Alternatively, the bristles 22 may be affixed to one or more collars or rings and then the collars or rings coupled to the outer surface of the elongate shaft 20. It is contemplated that a collar or ring may be a solid component or may be a plurality of wires twisted into a cylindrical tube in which the bristles 22 may be clamped between the twisted wires.

The bristles 22 may be employed to brush against a tissue surface in the target area 14 to capture cells. The bristles 22 may be arranged/mounted around the distal portion of the elongate shaft 20. In some embodiments, each of the bristles 22 may have a cross-section that is substantially circular. However, the bristles 22 may have any other suitable cross-sectional shape, including rectangular, triangular, square, polygonal, elliptical, or oblong.

The bristles 22 may be made of one or more filaments. For example, a plurality of the bristles 22 may be made of a continuous length of a filament. Alternatively, a plurality of the bristles 22 may be made of separate lengths of a filament. The filament may be a monofilament. The monofilament may be formed by extrusion. Alternatively, the filament may be a multicomponent filament. A multicomponent filament may include a core about which one or more layers of material are concentrically arranged. If multiple layers are present, they may differ in composition and/or thickness. The outermost one of the layers may include micropatterning. The multicomponent filament may be formed by coextrusion. It is contemplated that the filament may be made of nylon, polymer, and/or any suitable material or combination of materials.

It is contemplated that the bristles 22 may be arranged in any configuration desired. In some cases, the bristles 22 may be arranged helically around the distal portion of the elongate shaft 20 and may extend radially outwards from the elongate shaft 20. In some embodiments, the bristles 22 may radiate at an angle relative to the longitudinal axis of the elongate shaft 20 (e.g., at a generally orthogonal angle or a non-orthogonal angle). It is further contemplated that a density of the bristles 22 may vary along a length of the tissue collection device 12. In some cases, a length of the bristles 22 may varied. When the bristles 22 are brushed against tissue in the target area, cells from the tissue may be transferred to the bristles 22 and may be captured between the bristles 22.

When the tissue collection device 12 is disposed within the outer tubular member 18, the tissue collection device 12 may be restrained in a compressed reduced diameter or delivery configuration by the outer tubular member 18 surrounding the tissue collection device 12. In the compressed configuration, the tissue collection device 12 may have a smaller diameter than the expanded deployed configuration. The distal end region 26 of the outer tubular member 18 may be positioned such that the outer tubular member 18 surrounds and covers the length of the tissue collection device 12 during delivery. The outer tubular member 18 may have sufficient hoop strength to retain the tissue collection device 12 in its reduced diameter state.

The system 10 further include a filter 42 disposed within the lumen 24 of the outer tubular member 18. In some examples, the filter 42 may be coupled or affixed to an inner surface of the outer tubular member 18. In other examples, an outer surface of the filter 42 may form a friction fit with the inner surface of the outer tubular member 18. The filter 42 may include a lumen 44 extending from a proximal end 46 to a distal end 48 thereof. The lumen 44 may be sized to allow the elongate shaft 20 to freely slide therein. In some embodiments, the outer surface of the elongate shaft 20 may be coupled to an inner surface of the lumen 44 of the filter 42 such that the filter 42 moves with the elongate shaft 20.

The filter 42 may be formed from a sintered metal, a plastic mesh, a natural fiber mesh or cluster. The filter 42 may include a plurality of pores 50 sized to allow fluid, such as, but not limited to, blood to pass through the filter 42 while capturing cells dislodged from the target region 14 with the tissue collection device 12. In some cases, the pores 50 may have a size in the range of about 4 micrometers to about 9 micrometers. However, in some examples, at least a portion of the filter 42 may have a pore size of less than 4 micrometers or greater than 9 micrometers, as desired. In some examples, a vacuum source 54 may apply a vacuum to the proximal end region 28 of the outer tubular member 18 to draw fluid into the lumen 24 via the distal opening 38. As fluid is pulled into lumen 24, the fluid may pass through the filter 42. The filter 42 may trap cells and tissue while allowing fluid to pass therethrough. Cells and tissues may collect on the distal end 48 or distal end region of the filter 42 until the filter 42 is clogged with debris. When the filter 42 is clogged with debris, fluid may no longer be drawn through the lumen 24 of the outer tubular member 18. In some examples, the vacuum source 54 may be a syringe coupled to the port 32 of the hub 30 (or another location). The plunger of the syringe may be pulled away from the barrel to pull a vacuum on the lumen 24 of the outer tubular member 18. In other examples, the vacuum source 54 may be a vacuum pump coupled to the port 32 of the hub 30 (or another location).

In some embodiments, the plurality of pores 50 may all be substantially the same size. In other embodiments, the pore size of the plurality of pores may vary along a length of the filter 42. For example, in some embodiments, the pore size of the plurality of pores 50 may be larger at the distal end 48 of the filter 42 than the pore size of the pores 50 at the proximal end 46. This may allow larger particles to be trapped adjacent the distal end 48 or distal end region while allow smaller particles to travel further proximally within the filter 42 before becoming trapped. It is contemplated that such a gradation of pore size may allow more cells and/or tissue to accumulate in the filter 42 before the filter 42 becomes clogged. In some cases, the pore size of the plurality of pores 50 may gradually increase in size from the proximal end 46 of the filter 42 to the distal end 48 of the filter 42. In other examples, the pore size of the plurality of pores 50 may change in an abrupt or stair-step manner such that the filter 42 includes at least two regions of differently sized pores 50 with each region having a same size pore 50 throughout.

To collect a sample, the tissue collection system 10 may be advanced through the body towards the target location 14, as desired. The tissue collection system 10 may be advanced with or without the use of a guidewire. The tissue collection device 12 may be deployed by actuating the proximal end region 36 of the elongate shaft 20, for example, by distally pushing the proximal end region 36, while maintaining the first handle 30 in a fixed position. Thus, the elongate shaft 20 may be distally advanced relative to the outer tubular member 18. In other words, the elongate shaft 20 may be distally advanced while the outer tubular member 18 is held stationary. The reverse configuration is also contemplated. For example, the outer tubular member 18 may be proximally retracted while the elongate shaft 20 is held stationary. As the elongate shaft 20 is distally advanced, the biasing force is removed from the exterior of the tissue collection device 12 and the bristles 22 may assume their radially expanded, unbiased, deployed configuration (if the bristles 22 are compressed within the outer tubular member 18), shown in FIG. 1.

Once the tissue collection device 12 is deployed from the outer tubular member 18, the proximal end region 36 of the elongate shaft 20 may be actuated to repeatedly distally advance, proximally retract, and/or rotate the tissue collection device 12 along the target collection site 14. This may cause the bristles 22 to brush against the tissue surface to dislodge and capture cells. Some cells may be trapped between the bristles 22 while other cells 52 may be dislodged into the vessel lumen. As the elongate shaft 20 is actuated, the vacuum source 54 may be activated (e.g., the syringe plunger retracted, the pump activated, etc.) to draw bodily fluid into the lumen 24. As the bodily fluid is draw into the lumen 24, tissue and/or cells 52 dislodged by the tissue collection device 12 are also drawn into the lumen 24. The tissue and/or cells 52 may be trapped by the filter 42, as the bodily fluid is drawn proximally through the lumen 24. The vacuum source 54 may be activated or actuated for a predetermined length of time or until the filter 42 has become clogged with tissue/cells 52. In some embodiments, the amount of suction required to pull the vacuum may be used to determine when the filter 42 has become clogged. In some examples, the suction may be shut off or terminated based on a measured pressure or suction force prior to the filter 42 becoming completely clogged.

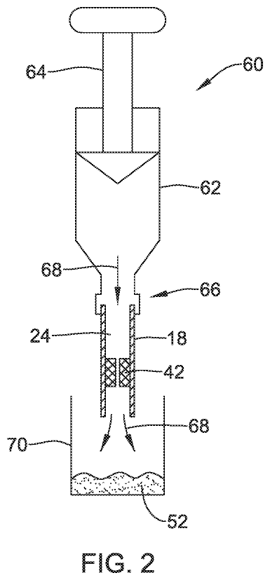

Once the filter 42 is clogged or a predetermined length of time has elapsed, the tissue collection system 10 may be removed from the body. It is contemplated that the bristles 22 may be retracted into the lumen 24 of the outer tubular member 18 prior to removal of the system 10 from the body. The elongate shaft 20 may be cut (e.g., using wire cutters or other cutting device) at a location proximal to the bristles 22 such that the bristles 22 may be placed into a sample container. To flush the tissue/cells 52 from the filter 42, the outer tubular member 18 may be coupled to a syringe or other flushing system. FIG. 2 is a schematic partial cross-sectional view of the outer tubular member 18 coupled to a syringe 60. The syringe 60 may include a barrel portion 62 for holding a quantity of liquid, a plunger 64 slidably disposed within a cavity of the barrel portion 62, and a coupling mechanism 66. The coupling mechanism 66 may be coupled to the outer tubular member 18 to fluidly couple the syringe 60 with the lumen 24 of the outer tubular member 18. In some examples, the coupling mechanism 66 may be a Tuohy Borst connection. However, this is not required. Other coupling mechanisms may be used, as desired. In some embodiments, the coupling mechanism 66 of the syringe 60 may be coupled with a proximal end region 28 of the outer tubular member 18. For example, the coupling mechanism 66 may be coupled to the hub 30 or the port 32. In other embodiments, the outer tubular member 18 may be cut at a location proximal to the filter 42 to create a shorter length of tube to be flushed. The coupling mechanism 66 may then be coupled to a proximal end of the shorter length of tube.

Once the syringe 60 is coupled to the outer tubular member 18, fluid 68 may be flushed out of the syringe 60 and through the lumen 24 of the outer tubular member 18. The fluid 68 may force the captured tissue/cells 52 out of the filter 42 and into a sample collection container 70. In some examples, the fluid may be saline. In other examples, the fluid may be the bodily fluid that was removed during sample collection. It is contemplated that in some cases, the sample collection device 12 may remain within the lumen 24 of the outer tubular member 18 during the flushing procedure. When the sample collection device 12 remains within the lumen 24, the fluid 68 may force the captured tissue/cells 52 out of the filter 42 as well as the bristles 22 of the tissue collection device 12 simultaneously. In yet another example, the filter 42 may be pushed out of the distal opening 38 of the outer tubular member 18 and placed in a sample collection container along with the bristles 22 of the sample collection device 12. It is contemplated that collecting tissue/cells 52 using both the bristles 22 and the filter 42 may result in a larger sample size than the use of a brush alone. This may allow the system 10 to collect a sufficient amount of sample for better prediction of biliary cancer (or other ailments).

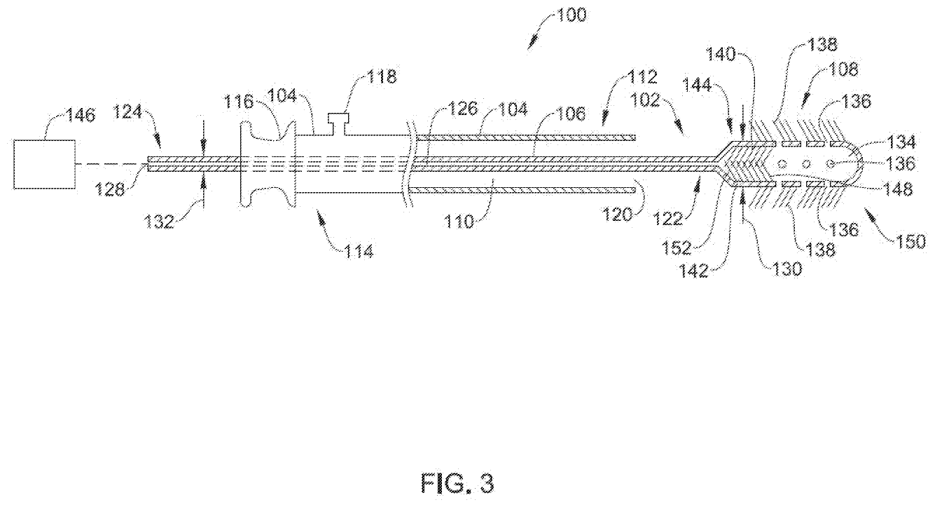

FIG. 3 is a partial cross-sectional side view of another illustrative tissue collection device system 100 for delivering a tissue collection device 102 to a target region, such as, but not limited to, the common bile duct or pancreatic duct, in an extended or use configuration. The tissue collection system 100 may include an outer or exterior elongate shaft or tubular member 104, such as, but not limited to, a catheter or introduction sheath. The system 100 may further include a tissue collection device 102 having an elongate tubular shaft 106 and a tissue collection member 108 disposed on a distal portion of the elongate tubular shaft 106. The tissue collection device 102 may be slidably disposed within a lumen 110 of the outer tubular member 104.

The outer tubular member 104 may extend proximally from a distal end region 112 to a proximal end region 114 configured to remain outside of a patient's body. A first hub or handle 116 may be coupled to the proximal end region 114 of the outer tubular member 104. In some cases, a port 118, such as an injection port, may be provided in the outer tubular member 104. Other structures to facilitate connection to other medical devices (e.g., syringe, stopcocks, Y-adapter, etc.) and to provide access to lumen 110 may be provided. The outer tubular member 104 may include a lumen 110 extending from a distal opening 120 at the distal end region 112 to the proximal end region 114. The lumen 110 may also extend through the first handle 116. The lumen 110 of the outer tubular member 104 and the first handle 116 may be configured to slidably receive the elongate tubular shaft 106. The lumen 110 of the outer tubular member 104 may also be configured to receive a guidewire (not explicitly shown). It is contemplated that the system 100 may be arranged such that the guidewire extends within a lumen along an entire length of the outer tubular member 104 in an “over-the-wire” manner or the guidewire may exit a side port in the outer tubular member 104 distal to the proximal end region 114 thereof in a “rapid-exchange” manner.

The elongate tubular shaft 106 may extend proximally from a distal end region 122 to a proximal end region 124 configured to remain outside of a patient's body. The elongate tubular shaft 106 may define a lumen 126 extending from a proximal opening 128 adjacent the proximal end region 124 to the distal end region 122. The tissue collection member 108 may be coupled to the distal end region 122 of the elongate tubular shaft 106. The tissue collection member 108 may have a diameter or cross-sectional dimension 130 that is greater than a diameter of the elongate tubular shaft 106. The tissue collection member 108 may include a cavity 134 in fluid communication with the lumen 126 of the elongate tubular shaft 106. The tissue collection member 108 may further include one or more apertures 136 extending through a thickness of the wall of the tissue collection member 108 such that the apertures 136 extend from the outer surface of the tissue collection member 108 to the cavity 134. The tissue collection member 108 may include any number of apertures 136 desired, such as, but not limited to, one, two, three, four, five, or more. In some examples, the tissue collection member 108 may include more than 10 apertures 136. It is contemplated that the number of apertures 136 may be determined by a size of the tissue collection member 108, a size of the apertures 136, and/or a desired use of the tissue collection device 102. The apertures 136 may be spaced about a circumference of the tissue collection member 108, if so desired. The apertures 136 may be spaced along an entire length of the tissue collection member 108 or extend along less than an entire length of the tissue collection member 108, as desired. In some embodiments, the apertures 136 may be positioned along a distal region 150 of the tissue collection member 108. In other embodiments, the apertures 136 may be positioned along a proximal region 144 of the tissue collection member 108. In some examples, the apertures 136 may be uniformly spaced. In other examples, the apertures 136 may be eccentrically positioned as desired. In yet other examples, at least one of the apertures 136 may be distally oriented. It is contemplated that the apertures 136 may have a diameter sufficient to allow tissue/cells to pass into the cavity 134 of the tissue collection member 108 via the apertures 136.

In some embodiments, the apertures 136 may have diameter (or major dimension) in the range of about 0.010 inches (0.254 millimeters) to about 0.200 inches (5.08 millimeters). It is contemplated that the size and/or shape of the apertures 136 may at least partially determine the number of apertures 136 required. For example, an aperture 136 having a major dimension in the range of about 0.200 inches (5.08 millimeters) may be a single aperture a one or more circumferential locations and having a generally ovular, elliptical, or rounded rectangular shape extending along a length of the tissue collection member 108. In another example, a plurality of apertures 136 having a diameter (or major dimension) in the range of about 0.010 inches (0.254 millimeters) may be provided in two or more rows each extending along a length of the tissue collection member 108 and spaced about a circumference of the tissue collection member 108. It is contemplated that the total surface area of the apertures 136 may be determined to achieve a desired pressure drop.

In some embodiments, the tissue collection device 102 may include a brush mechanism including a plurality of bristles 138 disposed on an outer surface of the tissue collection member 108. While the tissue collection device 102 is described as including a brush mechanism, it is contemplated that other tissue collection mechanisms may be used, as desired. The bristles 138 may extend radially outward from an outer surface of the tissue collection member 108. The bristles 138 may be coupled, adhered, or otherwise affixed to surface of the tissue collection member 108. In some cases, the bristles 138 may be coupled directly to the tissue collection member 108. Alternatively, the bristles 138 may be affixed to one or more collars or rings and then the collars or rings coupled to the outer surface of the tissue collection member 108. It is contemplated that a collar or ring may be a solid component or may be a plurality of wires twisted into a cylindrical tube in which the bristles 138 may be clamped between the twisted wires.

The bristles 138 may be employed to brush against a tissue surface in the target area to capture cells. The bristles 138 may be arranged/mounted around the distal portion of the elongate tubular shaft 106. In some embodiments, each of the bristles 138 may have a cross-section that is substantially circular. However, the bristles 138 may have any other suitable cross-sectional shape, including rectangular, triangular, square, polygonal, elliptical, or oblong.

The bristles 138 may be made of one or more filaments. For example, a plurality of the bristles 138 may be made of a continuous length of a filament. Alternatively, a plurality of the bristles 138 may be made of separate lengths of a filament. The filament may be a monofilament. The monofilament may be formed by extrusion. Alternatively, the filament may be a multicomponent filament. A multicomponent filament may include a core about which one or more layers of material are concentrically arranged. If multiple layers are present, they may differ in composition and/or thickness. The outermost one of the layers may include micropatterning. The multicomponent filament may be formed by coextrusion. It is contemplated that the filament may be made of nylon, polymer, and/or any suitable material or combination of materials.

It is contemplated that the bristles 138 may be arranged in any configuration desired. In some cases, the bristles 138 may be arranged helically around the tissue collection member 108 and may extend radially outwards from the tissue collection member 108. In some embodiments, the bristles 138 may radiate at an angle relative to the longitudinal axis of the tissue collection member 108 (e.g., at a generally orthogonal angle or a non-orthogonal angle). It is further contemplated that a density of the bristles 138 may vary along a length of the tissue collection member 108. In some cases, a length of the bristles 138 may varied. When the bristles 138 are brushed against tissue in the target area, cells from the tissue may be transferred to the bristles 138 and may be captured between the bristles 138.

The tissue collection member 108 may further include a filter 140 disposed within the cavity 134 thereof. It is contemplated that the filter 140 may extend along an entire length of the cavity 134 or less than an entire length of the cavity 134. In some examples, the filter 140 may be positioned adjacent to a proximal end region 144 of the tissue collection member 108 at a location proximal to the apertures 136. However, this is not required. In some examples, the filter 140 may be positioned longitudinally adjacent to at least one aperture 136. It is contemplated that the filter 140 may be coupled to an inner surface of the tissue collection member 108. However, this is not required. In other examples, an outer surface of the filter 140 may form a friction fit with the inner surface of the outer tubular member 104.

The filter 140 may be formed from a sintered metal, a plastic mesh, a natural fiber mesh or cluster. The filter 140 may include a plurality of pores 142 sized to allow fluid, such as, but not limited to, blood to pass through the filter 140 while capturing cells dislodged from the target region with the tissue collection device 102. In some examples, a vacuum source 146 may apply a vacuum to the proximal opening 128 of the elongate tubular shaft 106 to draw fluid into cavity 134 of the tissue collection member 108 via the plurality of apertures 136. As fluid is pulled into the cavity 134 and subsequently into the lumen 126 of the elongate tubular shaft 106, the fluid may pass through the filter 140. The filter 140 may trap cells and tissue while allowing fluid to pass therethrough. Cells and tissues may collect on a distal end 148 or distal end region of the filter 140 when the filter 140 is proximal to the apertures 136 until the filter 140 is clogged with debris. It is contemplated that when the filter 140 is longitudinally adjacent to the apertures 136, cells and tissues may collect along a length of the filter 140. When the filter 140 is clogged with debris, fluid may no longer be drawn through the cavity 134 and into the lumen 126 of the elongate tubular shaft 106. In some examples, the vacuum source 146 may be a syringe coupled to the proximal opening 128 of the elongate tubular shaft 106 (or another location, such as, but not limited to, a hub or port). The plunger of the syringe may be pulled away from the barrel to pull a vacuum on the lumen 126 of the elongate tubular shaft 106. In other examples, the vacuum source 146 may be a vacuum pump coupled to the proximal opening 128 of the elongate tubular shaft 106 (or another location, such as, but not limited to, a hub or port)

In some embodiments, the plurality of pores 142 may be all of substantially the same size. In other embodiments, the pore size of the plurality of pores may vary along a length of the filter 140. For example, in some embodiments, the pore size of the plurality of pores 142 may be larger at the distal end 148 of the filter 140 than the size of the pores 142 at the proximal end 152. This may allow larger particles to be trapped adjacent the distal end 148 or distal end region while allowing smaller particles to travel further proximally within the filter 140 before becoming trapped. It is contemplated that such a gradation of pore size may allow more cells and/or tissue to accumulate in the filter 140 before the filter 140 becomes clogged. In some cases, the pore size of the plurality of pores 142 may gradually increase in size from the proximal end 152 of the filter 140 to the distal end 148 of the filter 140. In other examples, the pore size of the plurality of pores 142 may change in an abrupt or stair-step manner such that the filter 140 includes at least two regions of differently sized pores 142 with each region having a same size pore 142 throughout.

The elongate tubular shaft 106 may have a substantially solid side wall. The elongate tubular shaft 106 may have sufficient flexibility to allow it to bend during insertion of the tissue collection device 102 into or withdrawal of the tissue collection device 102 from the subject's body. The elongate tubular shaft 106 may have sufficient rigidity so that pushing or pulling of the elongate tubular shaft 106 may cause extension or retraction, respectively, of the tissue collection device 102 from the outer tubular member 104. A proximal end region 124 of the elongate tubular shaft 106 may be gripped by a user such that the user may manually push or pull the elongate tubular shaft 106. While not explicitly shown, in some embodiments, the proximal end region 124 of the elongate tubular shaft 106 may include a handle or hub to facilitate actuation of the elongate tubular shaft 106. Dimensions of the elongate tubular shaft 106 may vary depending upon the subject's anatomy and/or the type of procedure being performed.

During insertion of the system 100 into the subject's body, or withdrawal of the system 100 from the subject's body, the tissue collection device 102 may be in a retracted position, with the elongate tubular shaft 106 pulled proximally to position the bristles 138 within the lumen 110 of the outer tubular member 104. The elongate tubular shaft 106 of the tissue collection device 102 may be pushed distally to move the tissue collection device to an extended position (shown in FIG. 3), with the bristles 138 extending out of the distal opening 120, so they are exposed from the outer tubular member 104.

When the tissue collection device 102 is disposed within the outer tubular member 104, the tissue collection device 102 may be restrained in a compressed reduced diameter or delivery configuration by the outer tubular member 104 surrounding the tissue collection device 102. In the compressed configuration, the tissue collection device 102 may have a smaller diameter than the expanded deployed configuration. The distal end region 112 of the outer tubular member 104 may be positioned such that the outer tubular member 104 surrounds and covers the length of the tissue collection device 102 during delivery. The outer tubular member 104 may have sufficient hoop strength to retain the tissue collection device 102 in its reduced diameter state.

To collect a sample, the tissue collection system 100 may be advanced through the body towards the target location, as desired. The tissue collection system 100 may be advanced with or without the use of a guidewire. The tissue collection device 102 may be deployed by actuating the proximal end region 124 of the elongate tubular shaft 106, for example, by distally pushing the proximal end region 124, while maintaining the first handle 116 in a fixed position. Thus, the elongate tubular shaft 106 may be distally advanced relative to the outer tubular member 104. In other words, the elongate tubular shaft 106 may be distally advanced while the outer tubular member 104 is held stationary. The reverse configuration is also contemplated. For example, the outer tubular member 104 may be proximally retracted while the elongate tubular shaft 106 is held stationary. As the elongate tubular shaft 106 is distally advanced, the biasing force is removed from the exterior of the tissue collection device 102 and the bristles 138 may assume their radially expanded, unbiased, deployed configuration (if the bristles 138 are compressed within the outer tubular member 104), shown in FIG. 3.

Once the tissue collection device 102 is deployed from the outer tubular member 104, the proximal end region 124 of the elongate tubular shaft 106 may be actuated to repeatedly distally advance, proximally retract, and/or rotate the tissue collection device 102 along the target collection site. This may cause the bristles 138 to brush against the tissue surface to dislodge and capture cells. Some cells may be trapped between the bristles 138 while other cells may be dislodged into the vessel lumen. As the elongate tubular shaft 106 is actuated, the vacuum source 146 may be activated (e.g., the syringe plunger retracted, the pump activated, etc.) to draw bodily fluid into the cavity 134 of the tissue collection member 108 and into the lumen 126 of the elongate tubular shaft 106. As the bodily fluid is draw into the cavity 134, tissue and/or cells dislodged by the tissue collection device 102 are also drawn into the cavity 134. The tissue and/or cells are trapped by the filter 140, as the bodily fluid is drawn proximally through the cavity 134 and into the lumen 126 of the elongate tubular shaft 106. The vacuum source 146 may be activated or actuated for a predetermined length of time or until the filter 140 has become clogged with tissue/cells. In some embodiments, the amount of suction required to pull the vacuum may be used to determine when the filter 140 has become clogged. In some examples, the suction may be shut off or terminated based on a measured pressure or suction force prior to the filter 140 becoming completely clogged.

Once the filter 140 is clogged or a predetermined length of time has elapsed, the tissue collection system 100 may be removed from the body. It is contemplated that the tissue collection member 108 may be proximally retracted into the lumen 110 of the outer tubular member 104 prior to removing the system 100 from the body. The elongate tubular shaft 106 may be cut (e.g., using wire cutters or other cutting device) at a location proximal to the bristles 138 such that the bristles 138 and the filter 140 may be placed into a sample container. In other embodiments, to flush the tissue/cells from the filter 140 and/or the bristles 138, the elongate tubular shaft 106 may be coupled to a syringe, in a manner similar to that shown and described with respect to FIG. 2. A coupling mechanism of a syringe may be coupled to the elongate tubular shaft 106 to fluidly couple the syringe with the lumen 126 of the elongate tubular shaft 106. In some examples, the coupling mechanism may be a Tuohy Borst connection. However, this is not required. Other coupling mechanisms may be used, as desired. In some embodiments, the coupling mechanism of the syringe may be coupled with a proximal end region 124 of the elongate tubular shaft 106. For example, the coupling mechanism may be coupled to the proximal opening 128 or a hub or port, if so provided. In other embodiments, the elongate tubular shaft 106 may be cut at a location proximal to the filter 140 to create a shorter length of tube. The coupling mechanism may then be coupled to a proximal end of the shorter length of tube.

Once the syringe is coupled to the elongate tubular shaft 106, fluid may be flushed out of the syringe and through the lumen 126 of the elongate tubular shaft 106. The fluid may force the captured tissue/cells out of the filter 140 and/or bristles 138 and into a sample collection container. In some examples, the fluid may be saline. In other examples, the fluid may be the bodily fluid that was removed during sample collection. It is contemplated that collecting tissue/cells using both the bristles 138 and the filter 140 may result in a larger sample size than the use of a brush alone. This may allow the system 100 to collect a sufficient amount of sample for better prediction of biliary cancer (or other ailments).

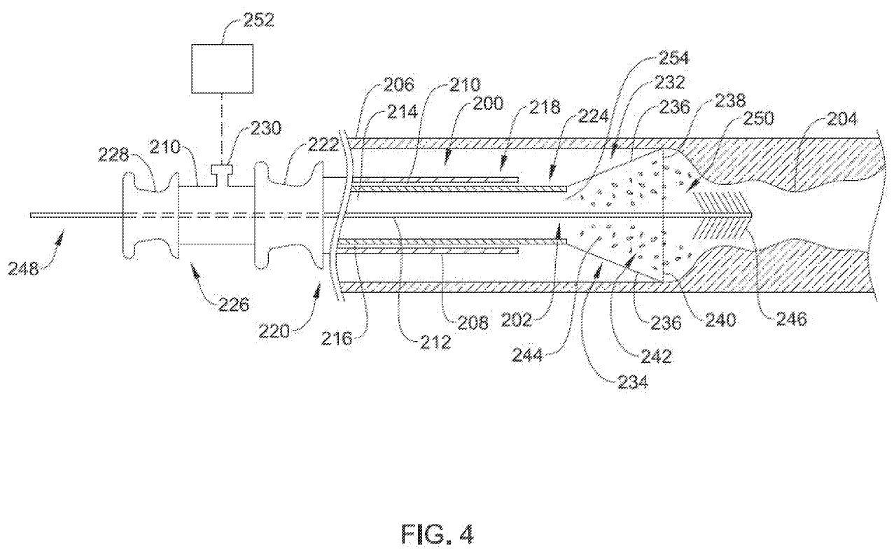

FIG. 4 is a partial cross-sectional side view of another illustrative tissue collection device system 200 for delivering a tissue collection device 202 to a target region 204, such as, but not limited to, the common bile duct or pancreatic duct 206, in an extended or use configuration. The tissue collection system 200 may include an outer or exterior elongate shaft or tubular member 208, such as, but not limited to, a catheter or introduction sheath and an inner tubular member 210. The system 200 may further include a tissue collection device 202 having an elongate shaft 212 and a plurality of bristles 246 disposed on a distal portion of the elongate shaft 212. The tissue collection device 202 may be slidably disposed within a lumen 214 of the inner tubular member 210.

The inner tubular member 210 may be slidably disposed within a lumen 216 of the outer tubular member 208. The outer tubular member 208 may extend proximally from a distal end region 218 to a proximal end region 220 configured to remain outside of a patient's body. A first hub or handle 222 may be coupled to the proximal end region 220 of the outer tubular member 208. In some cases, a port (not explicitly shown), such as an injection port, may be provided in the outer tubular member 208. Other structures to facilitate connection to other medical devices (e.g., syringe, stopcocks, Y-adapter, etc.) and to provide access to lumen 216 may be provided. The inner tubular member 210 may extend proximally from a distal end region 224 to a proximal end region 226 configured to remain outside of a patient's body. A second hub or handle 228 may be coupled to the proximal end region 226 of the inner tubular member 210. In some cases, a port 230, such as an injection port, may be provided in the inner tubular member 210. Other structures to facilitate connection to other medical devices (e.g., syringe, stopcocks, Y-adapter, etc.) and to provide access to lumen 214 may be provided.

The outer tubular member 208 may include a lumen 216 extending from the distal end region 218 to the proximal end region 220. The lumen 216 may also extend through the first handle 222. The lumen 216 of the outer tubular member 208 and the first handle 222 may be configured to slidably receive the inner tubular member 210. The inner tubular member 210 may include a lumen 214 extending from the distal end region 224 to the proximal end region 226. The lumen 214 of the inner tubular member 210 may also extend through the second handle 228. The lumen 214 of the inner tubular member 210 may be configured to receive a guidewire (not explicitly shown), as desired. It is contemplated that the system 200 may be arranged such that the guidewire extends within a lumen along an entire length of the inner tubular member 210 or outer tubular member 208 in an “over-the-wire” manner or the guidewire may exit a side port in the inner tubular member 210 or outer tubular member 208 distal to the proximal end regions 226, 220 thereof in a “rapid-exchange” manner.

The inner tubular member 210 may further include a distal filter assembly 232 extending distally from the distal end region 224 thereof. The filter assembly 232 may define an open cavity 254 with a distally facing opening 238 which is in fluid communication with the lumen 214 of the inner tubular member 210. In some examples, the distal filter assembly 232 may comprise a self-expanding filter assembly 232. For example, the distal filter assembly 232 may be compressed to fit within the outer tubular member 208 and upon proximal retraction of the outer tubular member 208 or distal advancement of the inner tubular member 210, the distal filter assembly 232 may expand without user intervention. Self-expandable members may be formed of any material or structure that is in a compressed state when force is applied and in an expanded state when force is released. Such members may be formed, for example, of shape memory alloys such as nitinol or any other self-expandable materials. When employing such shape-memory materials, the distal filter assembly 232 may include a frame 234 which may be heat set in the expanded state and then compressed to fit within the outer tubular member 208, for example. In another embodiment, a spring may be provided to effect expansion. It is contemplated that nickel-titanium alloys may enable kink-resistant folding and self-expansion. In other examples, magnetic alloys, metals, metal alloys, polymers, composites, etc. may be used to form the frame 234 of the distal filter assembly 232.

The frame 234 of the distal filter assembly 232 may include one or more generally longitudinally extending arms or prongs 236. The frame 234 may include any number of prongs 236 desired such as, but not limited to, one, two, three, four, or more. The prongs 236 may be uniformly or eccentrically spaced about a circumference of the filter assembly 232 as desired. The prongs 236 may extend at an angle from a longitudinal axis of the inner tubular member 210 such that in the expanded configuration, the prongs 236 extend radially outward to bring a distal end region of the distal filter assembly 232 into contact with the vessel wall. In some examples, the frame 234 may optionally further include a hoop 240 coupled to the distal ends of the prongs 236. The hoop 240 may be configured to appose the vessel wall. The distal filter assembly 232 may further include a microporous filter element 242 coupled to the frame 234. In some embodiments, the filter element 242 may be a polymer including a plurality of laser-drilled holes. In other embodiments, the filter element 242 may be a woven or braided structure formed from one or more filaments. The filament(s) may be formed from a metal, a metal alloy, a polymer, etc. The holes may be of a same size or differing sizes, as desired.

The frame 234 may generally provide expansion support to the filter element 242 in the expanded state. In the expanded state, the filter element 242 is configured to filter fluid (e.g., blood) flowing through the filter element 242 and to inhibit or prevent particles (e.g., cells or tissue) from flowing through the filter element 242 by capturing the particles in the filter element 242 and/or directing the tissue/cells into the lumen 214 of the inner tubular member 210. In some examples, the distal end of the lumen 214 may also include the filter element. It is contemplated that the filter element 242 may capture tissue or cells 244 dislodged by the tissue collection device 202. The frame 234 may configured to anchor the distal filter assembly 232 by engaging or apposing the inner walls of a lumen (e.g., blood vessel 206) in which the distal filter assembly 232 is expanded.

In some examples, a vacuum source 252 may apply a vacuum to the proximal end region 226 of the inner tubular member 210 to draw fluid into the lumen 214 via the distal opening 238 of the filter assembly 232. As fluid is pulled into lumen 214, the fluid may pass through the filter element 242 and/or into the lumen 214 of the inner tubular member 210. The filter element 242 may trap cells and tissue while allowing fluid to pass therethrough. Cells and tissues may collect on the distal side surface of the filter element 242 until the filter element 242 is clogged with debris. In some examples, the vacuum source 252 may be a syringe coupled to the port 230, the hub 228, or another location. The plunger of the syringe may be pulled away from the barrel to pull a vacuum on the lumen 214 of the inner tubular member 210. In other examples, the vacuum source 252 may be a vacuum pump coupled to the to the port 230, the hub 228, or another location. Additionally, or alternatively, in some examples, the inner tubular member 210 may be proximally retracted or distally advanced to capture tissue and/or cells 244.

The elongate shaft 212 may include one or more wires twisted into an elongated form. The elongate shaft 212 may have sufficient flexibility to allow it to bend during insertion of the tissue collection device 202 into or withdrawal of the tissue collection device 202 from the subject's body. The elongate shaft 212 may have sufficient rigidity so that pushing or pulling of the elongate shaft 212 may cause extension or retraction, respectively, of the tissue collection device 202 from the inner tubular member 210. A proximal end region 248 of the elongate shaft 212 may be gripped by a user such that the user may manually push or pull the elongate shaft 212. Dimensions of the elongate shaft 212 may vary depending upon the subject's anatomy and/or the type of procedure being performed.

In some embodiments, the tissue collection device 202 may include a brush mechanism including a plurality of bristles 246. While the tissue collection device 202 is described as a brush mechanism, it is contemplated that other tissue collection mechanisms may be used, as desired. The bristles 246 may extend radially outward from an outer surface of the elongate shaft 212. The bristles 246 may be coupled, adhered, or otherwise affixed to surface of the elongate shaft 212. In some cases, the bristles 246 may be coupled directly to the elongate shaft 212. In some examples, the bristles 246 may be clamped between the twisted wires of the elongate shaft 212. Alternatively, the bristles 246 may be affixed to one or more collars or rings and then the collars or rings coupled to the outer surface of the elongate shaft 212. It is contemplated that a collar or ring may be a solid component or may be a plurality of wires twisted into a cylindrical tube in which the bristles 246 may be clamped between the twisted wires.

The bristles 246 may be employed to brush against a tissue surface in the target area 204 to capture cells. The bristles 246 may be arranged/mounted around the distal portion 250 of the elongate shaft 212. In some embodiments, each of the bristles 246 may have a cross-section that is substantially circular. However, the bristles 246 may have any other suitable cross-sectional shape, including rectangular, triangular, square, polygonal, elliptical, or oblong.

The bristles 246 may be made of one or more filaments. For example, a plurality of the bristles 246 may be made of a continuous length of a filament. Alternatively, a plurality of the bristles 246 may be made of separate lengths of a filament. The filament may be a monofilament. The monofilament may be formed by extrusion. Alternatively, the filament may be a multicomponent filament. A multicomponent filament may include a core about which one or more layers of material are concentrically arranged. If multiple layers are present, they may differ in composition and/or thickness. The outermost one of the layers may include micropatterning. The multicomponent filament may be formed by coextrusion. It is contemplated that the filament may be made of nylon, polymer, and/or any suitable material or combination of materials.

It is contemplated that the bristles 246 may be arranged in any configuration desired. In some cases, the bristles 246 may be arranged helically around the distal portion of the elongate shaft 212 and may extend radially outwards from the elongate shaft 212. In some embodiments, the bristles 246 may radiate at an angle relative to the longitudinal axis of the elongate shaft 212 (e.g., at a generally orthogonal angle or a non-orthogonal angle). It is further contemplated that a density of the bristles 246 may vary along a length of the tissue collection device 202. In some cases, a length of the bristles 246 may varied. When the bristles 246 are brushed against tissue in the target area, cells from the tissue may be transferred to the bristles 246 and may be captured between the bristles 246.

When the tissue collection device 202 is disposed within the inner tubular member 210, the tissue collection device 202 may be restrained in a compressed reduced diameter or delivery configuration by the inner tubular member 210 surrounding the tissue collection device 202. In the compressed configuration, the tissue collection device 202 may have a smaller diameter than the expanded deployed configuration. The distal end region 224 of the inner tubular member 210 may be positioned such that the inner tubular member 210 surrounds and covers the length of the tissue collection device 202 during delivery. The inner tubular member 210 may have sufficient hoop strength to retain the tissue collection device 202 in its reduced diameter state. Similarly, in the delivery configuration (not explicitly shown), the outer tubular member 208 may be positioned such that the outer tubular member 208 surrounds and covers the length of the inner tubular member 210 during delivery. The outer tubular member 208 may have sufficient hoop strength to retain the filter assembly 232 in its reduced diameter state.

During insertion of the system 200 into the subject's body, or withdrawal of the system 200 from the subject's body, the tissue collection device 202 may be in a retracted position, with the elongate shaft 212 pulled proximally to position the bristles 246 within the lumen 214 of the inner tubular member 210. The elongate shaft 212 of the tissue collection device 202 may be pushed distally to move the tissue collection device to an extended position (shown in FIG. 4), with the bristles 246 extending out of the distal opening 238 of the filter assembly 232, so they are exposed from the inner tubular member 210.

To collect a sample, the tissue collection system 200 may be advanced through the body towards the target location 204, as desired. The tissue collection system 200 may be advanced with or without the use of a guidewire. The tissue collection device 202 may be deployed by actuating the proximal end region 248 of the elongate shaft 212, for example, by distally pushing the proximal end region 248, while maintaining the second handle 228 in a fixed position. Thus, the elongate shaft 212 may be distally advanced relative to the inner tubular member 210. In other words, the elongate shaft 212 may be distally advanced while the inner tubular member 210 is held stationary. The reverse configuration is also contemplated. For example, the inner tubular member 210 may be proximally retracted while the elongate shaft 212 is held stationary. As the elongate shaft 212 is distally advanced, the biasing force is removed from the exterior of the tissue collection device 202 and the bristles 246 may assume their radially expanded, unbiased, deployed configuration (if the bristles 246 are compressed within the inner tubular member 210), shown in FIG. 4. Further, the filter assembly 232 may be deployed by actuating the proximal end region 220 of the outer tubular member 208, for example, by distally retracting the proximal end region 220 while maintaining the second handle 228 in a fixed position. Thus, the outer tubular member 208 may be proximally retracted relative to the inner tubular member 210. In other words, the outer tubular member 208 may be proximally retracted while the inner tubular member 210 is held stationary. The reverse configuration is also contemplated. For example, the inner tubular member 210 may be distally advanced while the outer tubular member 208 is held stationary. As the outer tubular member is proximally retracted, the biasing force is removed from the exterior of the inner tubular member 210 and the filter assembly 232 may assume its radially expanded, unbiased, deployed configuration.

Once the tissue collection device 202 is deployed from the inner tubular member 210, the proximal end region 248 of the elongate shaft 212 may be actuated to repeatedly distally advance, proximally retract, and/or rotate the tissue collection device 202 along the target collection site 204. This may cause the bristles 246 to brush against the tissue surface to dislodge and capture cells. Some cells may be trapped between the bristles 246 while other cells 244 may be dislodged into the vessel lumen. As the elongate shaft 212 is actuated, the vacuum source 252 may be activated (e.g., the syringe plunger retracted, the pump activated, etc.) to draw bodily fluid into the lumen 214. As the bodily fluid is draw into the lumen 214, tissue and/or cells 244 dislodged by the tissue collection device 202 may also drawn into the lumen 214 of the inner tubular member 210. For example, if the filter element 242 does not extend across the diameter of the lumen 214 some tissue and/or cells may enter the lumen 214. In embodiments where the filter element 242 extends across the diameter of the lumen 214, the filter element 242 may trap the tissue and/or cells prior to the tissue and/or cells entering the lumen 214. The tissue and/or cells 244 are trapped by the filter element 242, as the bodily fluid is drawn proximally through the lumen 214. The vacuum source 252 may be activated or actuated for a predetermined length of time or until the filter element 242 has become clogged with tissue/cells 244. In some embodiments, the amount of suction required to pull the vacuum may be used to determine when the filter element 242 has become clogged.

Once the filter element 242 is clogged or a predetermined length of time has elapsed, the tissue collection system 200 may be removed from the body. In some examples, the elongate shaft 212 and the inner tubular member 210 may be retracted into the outer tubular member 208 for removal. It is contemplated that the elongate shaft 212 may be proximally retracted to positioned the bristles 246 within the cavity 254 of the filter assembly 232. The elongate shaft 212 and the inner tubular member 210 may then be proximally retracted substantially simultaneously such that the filter assembly 232 collapses over and generally surrounds the bristles 246. This may help prevent tissue and/or cells from becoming dislodged from the bristles 246 as the tissue collection device 202 is drawn into the inner tubular member 210.

The elongate shaft 212 may be cut (e.g., using wire cutters or other cutting device) at a location proximal to the bristles 246 such that the bristles 246 may be placed into a sample container. To flush the tissue/cells 244 from the filter element 242, the inner tubular member 210 may be coupled to a syringe, in a manner similar to that shown and described with respect to FIG. 2. A coupling mechanism of a syringe may be coupled to the inner tubular member 210 to fluidly couple the syringe with the lumen 214 of the inner tubular member 210. In some examples, the coupling mechanism may be a Tuohy Borst connection. However, this is not required. Other coupling mechanisms may be used, as desired. In some embodiments, the coupling mechanism of the syringe may be coupled with a proximal end region 226 of the inner tubular member 210. For example, the coupling mechanism may be coupled to a proximal opening, the hub 228, or port 230, if so provided. In other embodiments, the inner tubular member 210 may be cut at a location proximal to the filter element 242 to create a shorter length of tube. The coupling mechanism may then be coupled to a proximal end of the shorter length of tube.

Once the syringe is coupled to the inner tubular member 210, fluid may be flushed out of the syringe and through the lumen 214 of the inner tubular member 210. The fluid may force the captured tissue/cells out of the filter element 242 and into a sample collection container. In some examples, the fluid may be saline. In other examples, the fluid may be the bodily fluid that was removed during sample collection. In some examples, the filter element 242 may be rinsed to liberate the cells 244 without coupling the inner tubular member 210 to a syringe. It is contemplated that collecting tissue/cells 244 using both the bristles 246 and the filter element 242 may result in a larger sample size than the use of a brush alone. This may allow the system 200 to collect a sufficient amount of sample for better prediction of biliary cancer (or other ailments).

The materials that can be used for the various components of the medical device system 10, 100, 200 (and/or other systems disclosed herein) and the various elements thereof disclosed herein may include those commonly associated with medical devices. For simplicity purposes, the following discussion refers to the tissue collection system 10, 100, 200 and/or the tissue collection device 12, 102, 202. However, this is not intended to limit the devices and methods described herein, as the discussion may be applied to other elements, members, components, or devices disclosed herein, such as, but not limited to, the inner and outer tubular members 106, 210, 18, 104, 208, handles 30, 116, 222, 228, elongate shafts 20, 212, etc. and/or elements or components thereof.

In some embodiments, the tissue collection system 10, 100, 200, the tissue collection device 12, 102, 202, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, Marl MARLEX®ex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the tissue collection system 10, 100, 200, the tissue collection device 12, 102, 202, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the tissue collection system 10, 100, 200 and/or the tissue collection device 12, 102, 202 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the tissue collection system 10, 100, 200 and/or the tissue collection device 12, 102, 202 to achieve the same result.

In some embodiments, a degree of Magnetic Resonance Imaging (MRI) compatibility is imparted into the tissue collection system 10, 100, 200 and/or the tissue collection device 12, 102, 202. For example, the tissue collection system 10, 100, 200 and/or the tissue collection device 12, 102, 202, and/or components or portions thereof, may be made of a material that does not substantially distort the image and create substantial artifacts (e.g., gaps in the image). Certain ferromagnetic materials, for example, may not be suitable because they may create artifacts in an MRI image. The tissue collection system 10, 100, 200 and/or the tissue collection device 12, 102, 202, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, an exterior surface of the medical device system 10. 100, 200 (including, for example, an exterior surface of the delivery system) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the outer sheath, or in embodiments without an outer sheath over portions of the delivery system, or other portions of the medical device system 10. Hydrophobic coatings such as fluoropolymers provide a dry lubricity which improves device handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinyl alcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A tissue collection system, the system comprising:
a tubular member having a proximal end region and a distal end region and defining a lumen extending from the proximal end region to the distal end region;
an elongate shaft slidably disposed within the lumen of the tubular member, the elongate shaft extending from a proximal end region to a distal end region;
a tissue collection device disposed adjacent to the distal end region of the elongate shaft; and
a filter positioned adjacent to a distal end region of the tissue collection device when the tissue collection device is in a use configuration.

2. The tissue collection system of claim 1, wherein the filter includes a plurality of pores.

3. The tissue collection system of claim 2, wherein a pore size of the plurality of pores decreases from a distal end to a proximal end of the filter.

4. The tissue collection system of claim 1, wherein the filter is disposed within the lumen of the tubular member.

5. The tissue collection system of claim 4, wherein the filter defines a lumen extending from a proximal end to a distal end thereof.

6. The tissue collection system of claim 4, wherein the filter is coupled to an inner surface of the tubular member.

7. The tissue collection system claim 1, wherein the tissue collection device includes a tissue collection member defining a cavity.

8. The tissue collection system of claim 7, wherein the filter is disposed within the cavity of the tissue collection member.

9. The tissue collection system of claim 7, wherein the cavity of the tissue collection member is in fluid communication with a lumen of the elongate shaft.

10. The tissue collection system of claim 7, further comprising one or more apertures formed through a sidewall of the tissue collection member.

11. The tissue collection system of claim 1, wherein the filter comprises a filter assembly affixed to a distal end region of the tubular member.

12. The tissue collection system of claim 11, wherein the filter assembly includes an expandable frame and a filter element coupled to the expandable frame.

13. The tissue collection system of claim 11, further comprising an outer sheath, the tubular member slidably disposed within a lumen of the outer sheath.

14. The tissue collection system of claim 1, further comprising a vacuum source.

15. The tissue collection system of claim 14, wherein the vacuum source is configured to draw fluid and tissue through the filter.

16. A tissue collection system, the system comprising:
a tubular member having a proximal end region and a distal end region and defining a lumen extending from the proximal end region to the distal end region;
an elongate shaft slidably disposed within the lumen of the tubular member, the elongate shaft extending from a proximal end region to a distal end region;
a plurality of bristles disposed adjacent to the distal end region of the elongate shaft; and
a filter positioned within the lumen of the tubular member, the filter configured to collect cells dislodged from a target collection site.

17. The tissue collection system of claim 16, further comprising a vacuum source fluidly coupled to the proximal end region of the tubular member.

18. The tissue collection system of claim 17, wherein the vacuum source comprises a syringe.

19. A tissue collection system, the system comprising:
an outer tubular member having a proximal end region and a distal end region and defining a lumen extending from the proximal end region to the distal end region;
a tissue collection device comprising:
an elongate tubular shaft slidably disposed within the lumen of the outer tubular member, the elongate tubular shaft extending from a proximal end region to a distal end region;
a tissue collection member disposed on a distal end region of the elongate tubular shaft, the tissue collection member defining a cavity and having a plurality of apertures extending from an outer surface of the tissue collection member to the cavity; and
a plurality of bristles extending radially from an outer surface of the tissue collection member; and
a filter positioned within the cavity of the tissue collection member, the filter configured to collect cells dislodged from a target collection site.

20. The tissue collection system of claim 19, further comprising a vacuum source fluidly coupled to the proximal end region of the elongate tubular shaft.
